(19) United States
(12) Patent Application Publication
(10) Pub. No.: US 2018/0036071 A1
(43) Pub. Date: Feb. 8, 2018

(54) RADIOFREQUENCY BALLOON CATHETER SYSTEM

(71) Applicant: Japan Electel Inc., Kanagawa (JP)

(72) Inventor: Shutaro SATAKE, Kamakura-shi Kanagawa (JP)

(73) Assignee: Japan Electel Inc., Kanagawa (JP)

(21) Appl. No.: 15/542,224

(22) PCT Filed: Apr. 30, 2015
(86) PCT No.: PCT/JP2015/062989

§ 371 Date: Jul. 7, 2017

(51) Int. Cl. A61B 18/14; A61M 25/10

(52) U.S. Cl. CPC A61B 18/1492; A61M 25/1018; A61M 25/104; A61M 25/1002; A61B 2018/0022

(57) ABSTRACT

Provided is a radiofrequency balloon catheter system that allows inflation/deflation of the balloon in a controlled manner to enable the same to easily pass through the stenosis site without significantly changing the profile of a balloon (6). When a coolant is injected into the inside of the balloon (6), the balloon is inflated while discharging the coolant to the outside of the balloon (6), thus cooling the balloon (6) while applying a pressure to the balloon (6), with an intima being protected by perfusing the inside of the balloon (6) with a coolant. The radiofrequency balloon catheter system comprises a catheter shaft (1) comprising an inner tube shaft (3) and an outer tube shaft (2) that are slidable with each other; and a resilient balloon (6) that is inflatable and deflatable and provided between distal ends (4) of the inner tube shaft (3) and the outer tube shaft (2), said balloon (6) covering the inner tube shaft (3). The balloon (6) and the inner tube shaft (3) are in close proximity with each other to define a check valve (8). When the solution within the balloon (6) is suctioned to turn the pressure inside the balloon (6) to negative, the check valve (8) is closed and then the balloon (6) is deflated. Also, when a coolant is injected thereinto, the balloon (6) is inflated to open the check valve (8), thereby discharging the coolant to the outside. The radiofrequency balloon catheter system further includes an electrode for delivery of radiofrequency current (11) connected to a radiofrequency generator via a connecting wire (42) within said catheter shaft (1); and a solution transport path being in constant communication with an inside of the balloon (6) and connected to a liquid feed pump (29) for feeding a coolant (C).

### CROSS REFERENCE TO RELATED PATENT APPLICATIONS

This application is a U.S. National phase application under 35 U.S.C. 371 of International Patent Application No. PCT/JP2015/062989, filed Apr. 30, 2015. The International Application was published in Japanese on Nov. 3, 2016 as International Publication No. WO/2016174770 under PCT Article 21 (2).

### FIELD OF THE INVENTION

The present invention relates to a radiofrequency balloon catheter system for thermally dilating a stenosis by inserting a deflated balloon into the stenosis within a hollow organ, and irradiating the stenosis with a radiofrequency electric field power via an internal electrode while applying a pressure to the balloon, with an intima being protected by perfusing the inside of the balloon with a coolant.

### BACKGROUND OF THE INVENTION

Many of stenoses, such as coronary artery stenosis that cause angina or myocardial infarction are known to be due to arteriosclerotic lesions in a vascular membrane, and hence such stenoses are improved if they are dilated while applying a heat thereto using a radiofrequency hot balloon catheter. One example of ablation systems using such radiofrequency hot balloon catheter is disclosed in e.g., Patent document 1.

According to conventional radiofrequency hot balloon catheters, the balloon is deflated and inserted into a vascular stenosis site, then the balloon is pressurized and inflated to dilate the vascular stenosis, while heating the site by applying thereto a radiofrequency energy from an electrode inside the balloon to fuse collagen tissues and atheroma, etc. therein. Whilst such method as to dilate a vessel at a relatively low pressure while heating the vessel to soften and fuse a lesion therein has an advantage that the method does not cause vascular dissociation or recoil, and hence it is free from a risk of developing acute obstruction. The method, however, has a problem that restenosis may occur due to intimal proliferation caused by intimal ablation.

In order to prevent damages to an intima of a blood vessel, there have been developed balloon cooling methods using perfusion inside a balloon. Such methods include a method of perfusing a balloon interior through an outer tube and an inner tube of a catheter shaft, as disclosed in Patent Document 2, and a method of performing perfusion between the inside and the outside of a balloon through pores in a balloon film, as disclosed in patent Document 3, both of which were invented by the inventor of the present invention.

### PRIOR ART DOCUMENT

### Patent Document

Patent Document 1: Japanese Patent Application No. 2002-126096
Patent Document 2: U.S. Pat. No. 6,952,615
Patent Document 3: U.S. Pat. No. 6,491,710

### Problem to be Solved by the Invention

Among the systems to prevent damages to an intima of a blood vessel through an in-balloon perfusion system during heating using a radiofrequency balloon catheter, the system of Patent document 2, perfusing a balloon interior through the outer and inner tubes of a catheter shaft, exhibits an insufficient balloon cooling capacity due to a comparatively small amount of a perfusing solution resulting from a narrow shaft lumen for such a thin catheter as is used for coronary artery, etc.

According to the perfusion system of patent document 3 that discharges an in-balloon solution to the exterior through pores in a balloon film, passages between the balloon and the exterior are always open. For that reason, even if the solution is strongly suctioned from the inside of the balloon, the balloon does not fully deflate, making it difficult to insert the balloon catheter into a vascular stenosis site.

In view of the problems described above, it is an object of the present invention to provide a radiofrequency balloon catheter system employing a perfusion system with an enhanced balloon cooling capacity which discharges a solution within a balloon to the outside of the balloon, in which an ultra-compact check valve is provided in a path for discharging the in-balloon solution to the outside of the balloon, thereby allowing inflation/deflation of the balloon in a controlled manner without significantly changing the profile of the balloon, enabling the same to easily pass through the stenosis site.

### SUMMARY OF THE INVENTION

### Means for Solving the Problems

According to the radiofrequency balloon catheter system of the present invention, the anterior neck of the balloon is not fixed on the inner tube. Rather, the inner tube is covered with the anterior neck to be in contact with each other when the balloon is subject to a negative internal pressure to thereby define a check valve with a pair of the anterior neck and the inner tube for performing fluid control. That is, the flow is interrupted upon a contact of the anterior neck with the inner neck, but the flow resumes on the opening of a clearance gap between the anterior neck and the inner tube. Accordingly, when a coolant is injected into the balloon, the pressure within the balloon turns to positive to thereby inflate the balloon. If the in-balloon pressure exceed a given value (or cracking pressure), the valve defined by the anterior neck and the inner tube opens, thus discharging the solution therein to the outside of the balloon to thereby cool the balloon. The cracking pressure depends on the degree of tightness of the anterior neck of the balloon against the inner tube, which is dependent on the elasticity, inner diameter, and/or shape of the anterior neck. When the in-balloon solution is suctioned from the balloon to turn the pressure within the balloon to negative, the check valve is closed and then the balloon is deflated, thereby enabling insertion into the stenosis site to be easily performed, thus providing a solution to the above-described problems.

Since the present balloon cooling system utilizes conventional balloon catheter members, the system can be also applied to small diameter catheters such as those for coronary angioplasty without significantly changing the balloon profile.

According to a first aspect of the present invention, there is provided a radiofrequency balloon catheter system including:

a catheter shaft comprising an inner tube and an outer tube that are slidable with each other;

a resilient balloon that is inflatable and deflatable and provided between distal ends of the inner tube and the outer tube, said balloon including an anterior neck covering the inner tube;

a check valve defined by the inner tube and the anterior neck covering the inner tube, said anterior neck and said inner tube defining a clearance gap that opens when the balloon is subject to a positive internal pressure, while the anterior neck and the inner tube are contacted with each other to thereby close the clearance gap when the balloon is subject to a negative internal pressure;

an electrode for delivery of radiofrequency current provided within the balloon;

a radiofrequency generator connected to the electrode for delivery of radiofrequency current via a connecting wire within said catheter shaft; and

a solution transport path defined by the outer tube and the inner tube, said solution transport path being in communication with an inside of the balloon, and connected to a liquid feed pump for feeding a coolant, as illustrated in FIGS. 1 to 4.

A second aspect of the present invention is a radiofrequency balloon catheter system based on the first aspect, wherein a distal portion of the inner tube is reduced in a tapered manner to adjust the size of the clearance gap between the anterior neck and the inner tube by sliding the inner tube against the outer tube, as illustrated in FIG. 5.

A third aspect of the present invention is a radiofrequency balloon catheter system based on the first aspect, wherein a distal portion of the inner tube is dilated to have such an enlarged diameter portion that allows the clearance gap between the anterior neck and the inner tube to be adjustable in size by sliding the inner tube against the outer tube, as illustrated in FIG. 6.

A fourth aspect of the present invention is a radiofrequency balloon catheter system based on the first aspect, wherein a distal portion of the inner tube is perforated with small holes to adjust the discharge rate of a coolant from the inside of the balloon by sliding the inner tube against the outer tube to change the extent of an overlap between the anterior neck and the small holes of the distal portion of the inner tube, as illustrated in FIG. 7.

A fifth aspect of the present invention is a radiofrequency balloon catheter system based on the first aspect, wherein the anterior neck of the balloon is provided with slits or perforated with small holes to readily open the clearance gap, defined by said anterior neck and said inner tube, to be in communication with the outside of the balloon when the balloon is subject to a positive internal pressure, as illustrated in FIG. 8.

A sixth aspect of the present invention is a radiofrequency balloon catheter system based on the first aspect, wherein a temperature sensor and a pressure sensor are installed within said balloon and are respectively connected to a temperature measurement device and a pressure measurement device via a connecting wire, as illustrated in FIG. 9.

A seventh aspect of the present invention is a radiofrequency balloon catheter system based on the first aspect, wherein electrodes are respectively installed in front and back of said balloon on said catheter shaft, and the electrodes are connected to an impedance measurement device via connecting wire, as illustrated in FIG. 9.

### Effects of the Present Invention

Referring to FIG. 1A showing a schematic diagram of the present invention in accordance with the first aspect of the invention, when the solution within the balloon is suctioned via the catheter shaft, the check valve defined by the anterior neck and the inner tube is closed, thereby turning the pressure inside the balloon to negative, as shown in FIG. 1B. When the solution within the balloon is further suctioned, the balloon is deflated and then inserted into the stenosis site, as shown in FIG. 2. When a coolant is injected into the inside of the balloon, the balloon is inflated to open the check valve, thereby discharging the coolant to the outside of the balloon, thus cooling the balloon, as shown in FIG. 3. Discharge rate of the coolant depends on injection rate of the coolant to be injected into the balloon, and further on the elasticity and/or shape of the anterior neck of the balloon serving as a valving element. Further, an “overlap” between the anterior neck of the balloon, in communication with the outer tube, and the inner tube define a check valve that allows adjustment of the discharge rate by sliding the inner tube against the outer tube to change the extent of the “overlap”, as shown in FIGS. 1C and 1D. Concurrently therewith, upon delivery of radiofrequency current, a radio frequency electric field is radiated uniformly from the electrode for delivery of radiofrequency current arranged inside the balloon, thereby allowing the balloon to dilate the stenosis site by increasing an in-balloon pressure while heating the site. In the meantime, cooling of the balloon protects intima against heating, as illustrated in FIG. 4.

According to the first aspect of the invention, there can be provided a radiofrequency balloon catheter system enabling a balloon catheter thereof to easily pass through a stenosis and dilate the stenosis while heating the same, with an intima being protected against damage.

According to the second aspect of the invention, when the inner tube slides forward against the anterior neck of the balloon, the clearance gap between the inner tube and the anterior neck of the balloon become narrower to thereby decrease the discharge rate of the in-balloon solution. Meanwhile, when the inner tube slides backward against the anterior neck of the balloon, the clearance gap between the inner tube and the anterior neck of the balloon become wider to thereby increase the discharge rate of the in-balloon solution, as illustrated in FIGS. 5A and 5B.

According to the third aspect of the invention, when the inner tube slides backward against the anterior neck of the balloon, the clearance gap between the inner tube and the anterior neck of the balloon become narrower to thereby decrease the discharge rate of the in-balloon solution. Meanwhile, when the inner tube slides forward against the anterior neck of the balloon, the clearance gap between the inner tube and the anterior neck of the balloon become wider to thereby increase the discharge rate of the in-balloon solution, as illustrated in FIGS. 6A and 6B.

According to the fourth aspect of the invention, when the small holes in the inner tube slide backward to a position posterior to the anterior neck of the balloon, the small holes become cleared of the anterior neck to thereby increase the discharge rate of the in-balloon solution. Meanwhile, when the small holes in the inner tube slide forward to be faced against the anterior neck of the balloon, the small holes become covered by the anterior neck to thereby decrease the discharge rate of the in-balloon solution, as illustrated in FIG. 7.

According to the fifth aspect of the invention, the anterior neck of the balloon may has slits or otherwise perforated with small holes for letting the in-balloon solution be easily passed through the clearance gap between the inner tube and the anterior neck, thereby enhancing cooling capacity thereof, as illustrated in FIG. 8.

According to the sixth aspect of the invention, a temperature sensor and a pressure sensor are installed within the balloon, making it possible to monitor a balloon temperature and a pressing force of the balloon against tissues, thereby enabling one to make sure that ablation of a target tissue has been successfully done, as illustrated in FIG. 9.

According to the seventh aspect of the invention, electrodes are respectively installed in front and back of the balloon, making it possible to monitor an impedance around the balloon, thereby enabling one to follow up the extent of ablation of a target tissue, as illustrated in FIG. 9.

### BRIEF DESCRIPTION OF THE DRAWING FIGURES

FIG. 1A is an explanatory drawing illustrating a main framework of a radiofrequency balloon catheter system of the present invention, in which a balloon is additionally provided, at its distal end, with a check valve structure for an in-balloon perfusion system, the check valve defined by a balloon anterior neck and an inner tube.

FIG. 1B is an explanatory drawing illustrating a mechanism for deflating a balloon due to a closure of a check valve defined by the balloon anterior neck and the inner tube when suctioning an in-balloon solution according to a radiofrequency balloon catheter system of the present invention.

FIG. 1C is an explanatory drawing illustrating a mechanism for adjusting a discharging amount of an in-balloon solution by sliding the inner tube shaft against an outer tube shaft to change the extent of the overlap between the inner tube shaft and the anterior neck of the balloon defining a check valve. The drawing illustrates an arrangement of the inner tube and the outer tube shaft that are overlapped with each other.

FIG. 1D is an explanatory drawing illustrating a mechanism for adjusting a discharging amount of an in-balloon solution by sliding the inner tube shaft against the outer tube shaft to change the extent of the overlap between the inner tube shaft and the anterior neck of the balloon defining a check valve according to a radiofrequency balloon catheter system of the present invention. The drawing illustrates an arrangement of the inner tube and the outer tube shaft that are displaced from each other.

FIG. 2 is an explanatory drawing illustrating a balloon catheter being inserted into a stenosis using a guide wire after the balloon is deflated by strongly suctioning the inside of the balloon.

FIG. 3 is an explanatory drawing illustrating the stenosis being heated by irradiation of a radiofrequency field while allowing the inside of the balloon to be perfused with a coolant, after delivery of a radiofrequency current is started with the balloon being inflated by injecting the coolant thereinto.

FIG. 4 is an explanatory drawing illustrating the stenosis being dilated by further increasing an in-balloon pressure by raising an injection speed of the coolant.

FIG. 5A is an explanatory drawing illustrating a balloon catheter in which a distal portion of the inner tube shaft is tapered and has such a reduced diameter for adjusting a discharging amount of an inner solution by sliding the inner tube shaft against the outer tube shaft to vary a clearance gap between the anterior neck and the inner tube. The figure illustrates the balloon catheter discharging a lesser amount of inner solution.

FIG. 5B is an explanatory drawing illustrating a balloon catheter in which a distal portion of the inner tube shaft has a reduced diameter portion that is reduced in a tapered manner for adjusting a discharging amount of an inner solution by sliding the inner tube shaft against the outer tube shaft to vary a clearance gap between the anterior neck and the inner tube shaft. The figure illustrates the balloon catheter discharging a larger amount of inner solution.

FIG. 6A is an explanatory drawing illustrating a balloon catheter in which a distal portion of the inner tube shaft has such an enlarged portion for adjusting a discharging amount of an inner solution by sliding the inner tube shaft against the outer tube shaft to vary a clearance gap between the anterior neck and the inner tube shaft. The figure illustrates the balloon catheter discharging a lesser amount of inner solution.

FIG. 6B is an explanatory drawing illustrating a balloon catheter in which a distal portion of the inner tube has such an enlarged portion for adjusting a discharging amount of an inner solution by sliding the inner tube shaft against the outer tube shaft to vary a clearance gap between the anterior neck and the inner tube shaft. The figure illustrates the balloon catheter discharging a larger amount of inner solution.

FIG. 7A is an explanatory drawing illustrating a balloon catheter in which a distal portion of the inner tube shaft is perforated with small holes. The figure illustrates how a discharging amount of an inner solution is reduced by sliding the inner tube shaft forward against the outer tube shaft to cover the holes of the inner tube with the anterior neck of the balloon.

FIG. 7B is an explanatory drawing illustrating a balloon catheter in which a distal portion of the inner tube shaft is perforated with small holes. The figure illustrates how a discharging amount of an inner solution is increased by sliding the inner tube shaft backward against the outer tube shaft to let the holes in direct communication with the inside of the balloon.

FIG. 8 is an explanatory drawing illustrating a balloon catheter in which a distal portion of an outer tube shaft has slits or perforated with small holes. The figure illustrates how an in-balloon solution is easily discharged even when a distal portion of the anterior neck 6A is fixed to the outer tube owing to a proximal portion of the anterior neck serving as a check valve.

FIG. 9 is an explanatory drawing illustrating another framework where a temperature sensor and a pressure sensor are installed at distal portions of the inner tube within the balloon, thus enabling the measurement of a temperature of the in-balloon solution and a pressure inside the balloon, while electrodes are attached to the vicinity of the distal ends of the inner tube and the outer tube such as to sandwich the balloon with the same, thus enabling the measurement of an impedance across the front and rear of the balloon.

### DESCRIPTION OF EMBODIMENTS

As follows is a detailed description of embodiments of a radiofrequency balloon catheter system proposed by the present invention with reference to the appended drawings.

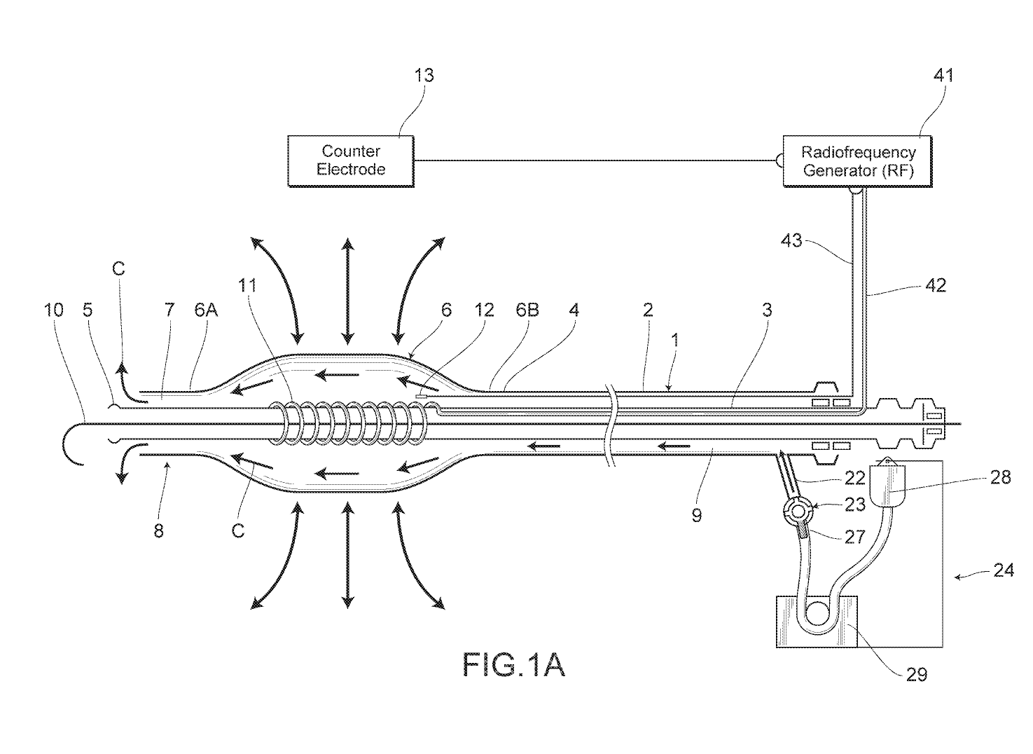

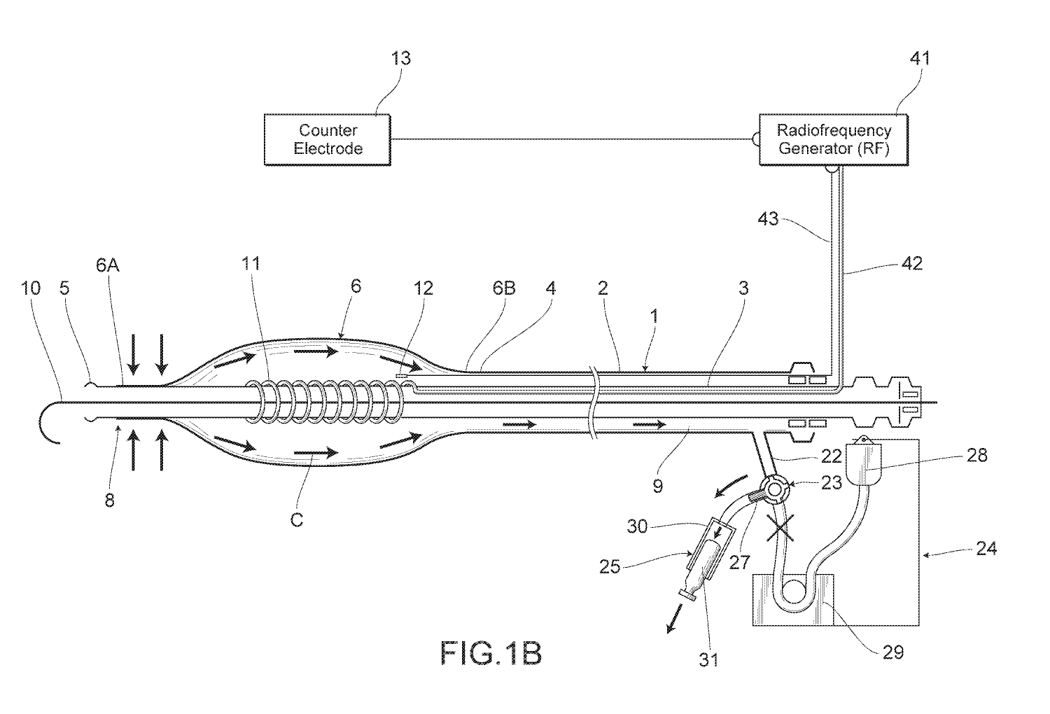

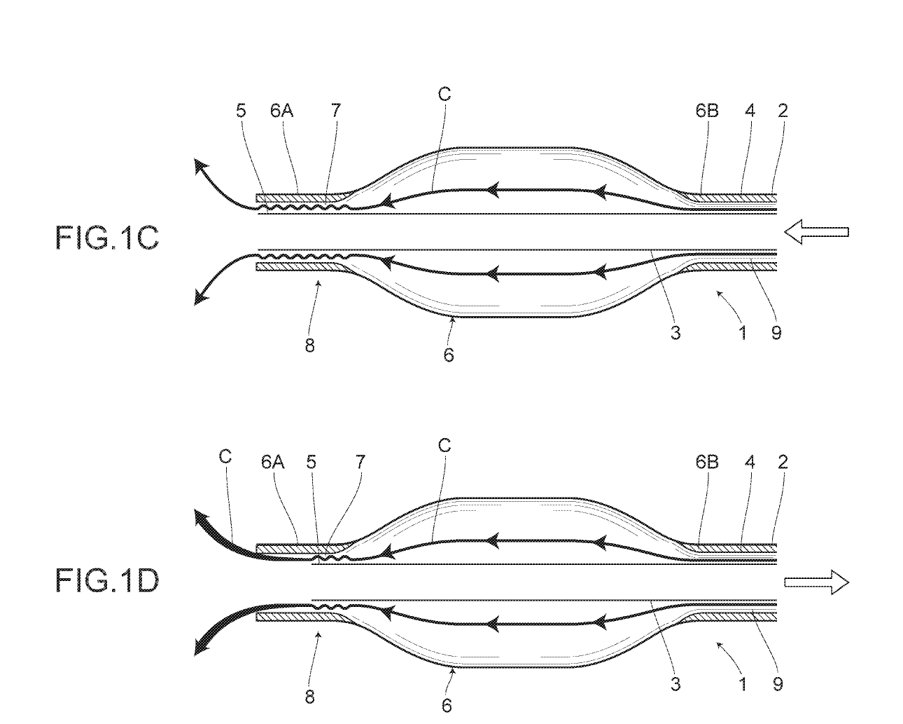

FIGS. 1A to 1B illustrate a major part structure of the radiofrequency balloon catheter system according to an embodiment of the present invention. In the drawings, numerical symbol 1 denotes a cylindrical catheter shaft that is rich in elasticity and insertable into a luminal organ. The catheter shaft 1 includes an outer tube shaft 2 and an inner tube shaft 3 which are hollow and slidable with each other in a longitudinal direction. A deflatable and inflatable balloon 6 is provided between a distal end 4 of the outer tube shaft 2 and a vicinity of a distal end 5 of the inner tube shaft 3. The balloon 6 is made of a thin membrane, which is formed of a heat-resistant resin such as polyurethane, PET (polyethylene terephthalate) or the like. The balloon 6 has an appropriate elasticity, and contains necks 6A and 6B respectively arranged in the anterior and posterior portions of the balloon 6, the necks 6A and 6B having a thickness thinner than any other portions of the balloon. The balloon 6 is allowed to inflate in the shape of a rotating body, e.g., substantially spherical shape, by filling a solution as a coolant C (normally, a cooled mixture of a non-ionic contrast agent and one of distillated water and dextrose in water) in the balloon 6.

Between the outer tube shaft 2 and the inner tube shaft 3 is defined a solution transport path 9 in communication with the inside of the balloon 6. The anterior neck 6A of the balloon 6 is not fixed to the inner tube shaft. As illustrated in FIG. 1A, when the balloon 6 is subjected to a positive pressure, between the anterior neck 6A and the inner tube shaft 3 is defined a clearance gap 7 for allowing the coolant C to be discharged from the inside of the balloon 6 to the outside of the balloon. In contrast to that, as illustrated in FIG. 1B, when the balloon 6 is subjected to a negative pressure, the anterior neck 6A is deformed and comes in contact with the inner tube shaft 3 to permit the flow of the coolant C in one direction. In this way, the anterior neck 6A and the inner tube shaft 3 respectively serve as a valving element of the check valve 8 and a valve seat of the check valve 8. Meanwhile, the posterior neck 6B of the balloon 6 is fixed to or continuously provided on the distal end 4 of the outer tube shaft 2. Numeral 10 denotes a guide wire for guiding the balloon 6 to a target site. The guide wire 10 is provided within the inner tube shaft 3 in a manner extending therethrough.

Inside the balloon 6 are arranged an electrode 11 for delivery of radiofrequency current and a temperature sensor 12. The electrode 11 for delivery of radiofrequency current is arranged, as an electrode for radiating a radiofrequency electric field, in such a coiled fashion that it is wound around the inner tube shaft 3. Further, the electrode 11 for delivery of radiofrequency current has a monopolar structure, and is able to deliver a radiofrequency current between itself and a counter electrode 13 provided outside the catheter shaft 1. When a current is applied thereto, then, there will be radiated an electric field from the electrode 11 for delivery of radiofrequency current to the surroundings thereof.

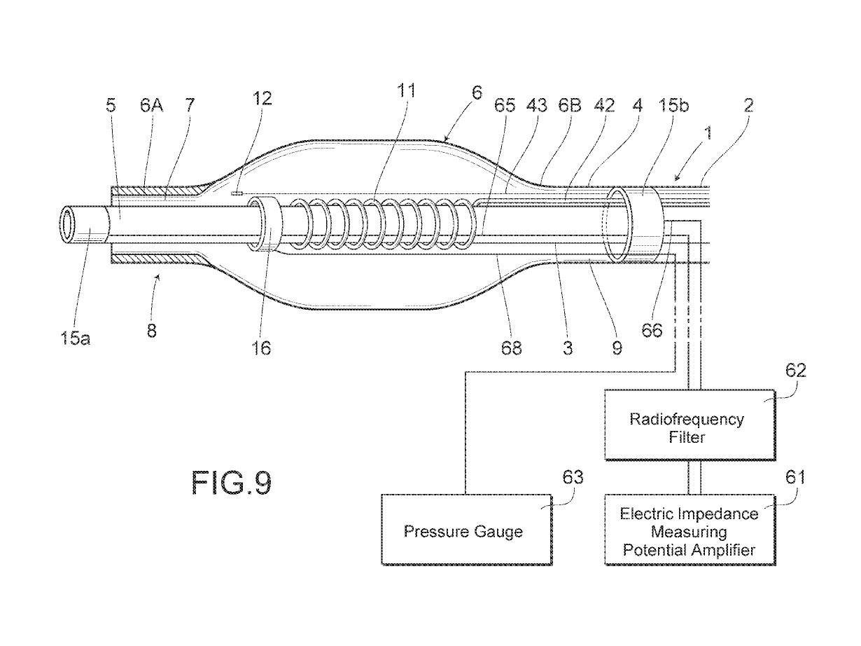

A temperature sensor 12, serving as a temperature detection unit, is provided on the proximal end side of the inner tube shaft 3 inside the balloon 6, and arranged in close proximity to the electrode 11 for delivery of radiofrequency current to detect the temperature of the electrode 11. Further, as illustrated in FIG. 9, there can be fixed not only the temperature sensor 12 but also electrodes 15a and 15b that are respectively provided on the anterior and posterior portions of the balloon 6 in order to measure the impedance therebetween. Further, in proximity to a front surface of the membrane inside the balloon 6, there may be provided a high directional pressure sensor 16 coaxially with the catheter shaft 1 with an input surface thereof facing forward in a longitudinal direction of the shaft 1.

Outside the catheter shaft 1, a communication tube 22 is connected to a basal portion of the solution transport path 9 in a communicative manner. One port of a three-way cock 23 is coupled to the basal portion of this communication tube 22, and the remaining two ports of the three-way cock 23 are respectively coupled to a liquid transfusing unit 24 for inflating the balloon 6 and a syringe 25 for deflating the balloon 6. The three-way cock 23 has an operation piece 27 capable of being pivotally operated by the fingers such that one of the liquid transfusing unit 24 and the syringe 25 may come into a fluid communication with the communication tube 22, or eventually with the solution transport path 9 by the operation of the operation piece 27.

The liquid transfusing unit 24 is made up of: an infusion bottle 28 for reserving the coolant C; and a liquid transfusing pump 29 in communication with the infusion bottle 28. When the liquid transfusing pump 29 is activated with the liquid transfusing unit 24 and the communication tube 22 communicated with each other through the three-way cock 23, the coolant C, having reached there from the infusion bottle 28, is pumped out into the solution transport path 9 through the liquid transfusing pump 29, thereby turning the pressure at the inside of the balloon 6 to positive. A syringe 25, serving as a liquid recovering unit, includes a cylindrical body 30, connected to the three-way cock 23, and a movable plunger 31 provided within the cylindrical body 30. If the plunger 31 is pulled back with the syringe 25 being communicated with the communication tube 22 through the three-way cock 23, the solution is recovered from the inside of the balloon 6 via the solution transport path 9 into the inside of the cylindrical body 28, thereby turning the pressure inside the balloon to negative.

Further, a radiofrequency generator 41 is provided outside of the catheter shaft 1. Within the balloon 6 are arranged the electrode 11 for delivery of radiofrequency current and the temperature sensor 12, which are electrically connected to the radiofrequency generator 41 respectively through the electric wires 42, 43 placed inside the catheter shaft 1. The radiofrequency generator 41 supplies a radiofrequency energy, to be delivered as an electric power, to between the electrode 11 for delivery of radiofrequency current and the counter electrode 13 through the electric wire 42, and heats the whole of the balloon 6 filled with the solution. The radiofrequency generator 41 is provided with a temperature indicator system (not shown) for measuring and displaying the temperature of the electrode 11 for delivery of radiofrequency current, and eventually, the internal temperature inside the balloon 6, through a detection signal from the temperature sensor 12 transmitted through the electric wire 43. Further, the radiofrequency generator 41 sequentially retrieves information on temperatures measured by the temperature indicator system to determine a level of a radiofrequency energy to be supplied through the electric wire 42 to between the electrode 11 for delivery of radiofrequency current and the counter electrode 13. The electric wires 42, 43 are fixed along the inner tube shaft 3 over the entire axial length of the inner tube shaft 3.

According to the present embodiment, whilst the electrode 11 for delivery of radiofrequency current is used as a heating means for heating the inside of the balloon 6, it is not to be limited to any specific ones as long as it is capable of heating the inside of the balloon 6. For example, as substitute for the electrode 11 for delivery of radiofrequency current and the radiofrequency generator 41, there may be employed any one of couples of: an ultrasonic heating element and an ultrasonic generator; a laser heating element and a laser generator; a diode heating element and a diode power supply; and a nichrome wire heating element and a nichrome wire power supply unit.

Further, the catheter shaft 1 and the balloon 6 are made of such a heat resistant resin that can withstand heating without causing thermal deformation and the like when heating the inside of the balloon 6. The balloon 6 may take not only a spherical shape whose long and short axes are equal, but also any other shapes of any rotational bodies such as an oblate spherical shape whose short axis is defined as a rotation axis, a prolate spheroid whose long axis is defined as a rotation axis, or a bale shape. In any of these shapes, the balloon is made up of such an elastic member having compliance that deforms when it comes in close contact with an inside wall of a luminal organ.

When the balloon is subject to a positive pressure as described above, the amount of the coolant C to be discharged through a clearance gap 8 of the check valve 7 to the outside of the balloon 6, that is, discharge rate of the solution from the inside of the balloon 6 can be adjusted by the extent of in-and-out operation of inner tube shaft 3 to the outer tube shaft 2. FIGS. 1C and 1D illustrate such operation. In each of the figures, the white hollow arrow, shown on the right, indicates a moving direction of the inner tube shaft 3.

If the inner tube shaft 3 slides in a axial direction against the outer tube shaft 2 to overlap the inner tube shaft 3 with the anterior neck 6A so as to arrange the distal opening section of the inner tube shaft 3 to be substantially flush with the distal opening section of the balloon 6, as illustrated in FIG. 1C, for example, then, most of the inner surface of the anterior neck 6A is entirely faced against the outer surface of the inner shaft 3, thereby decreasing the discharge rate of the coolant C passing through the clearance gap 7. In contrast, as illustrated in FIG. 1D, if the inner tube shaft 3 and the anterior neck 6A are displaced against each other so as to arrange the distal opening section of the inner tube shaft 3 in a position posterior to the distal opening section of the balloon 6, then, inner surface of the anterior neck 6A is partially faced against the outer surface of the inner shaft 3, thereby decreasing the discharge rate of the coolant C passing through the clearance gap 7. Consequently, as long as the check valve 8 is open, discharge rate of the solution inside the balloon 6 can be easily adjusted in accordance with a sliding amount of the inner tube shaft 3 against the outer tube shaft 2.

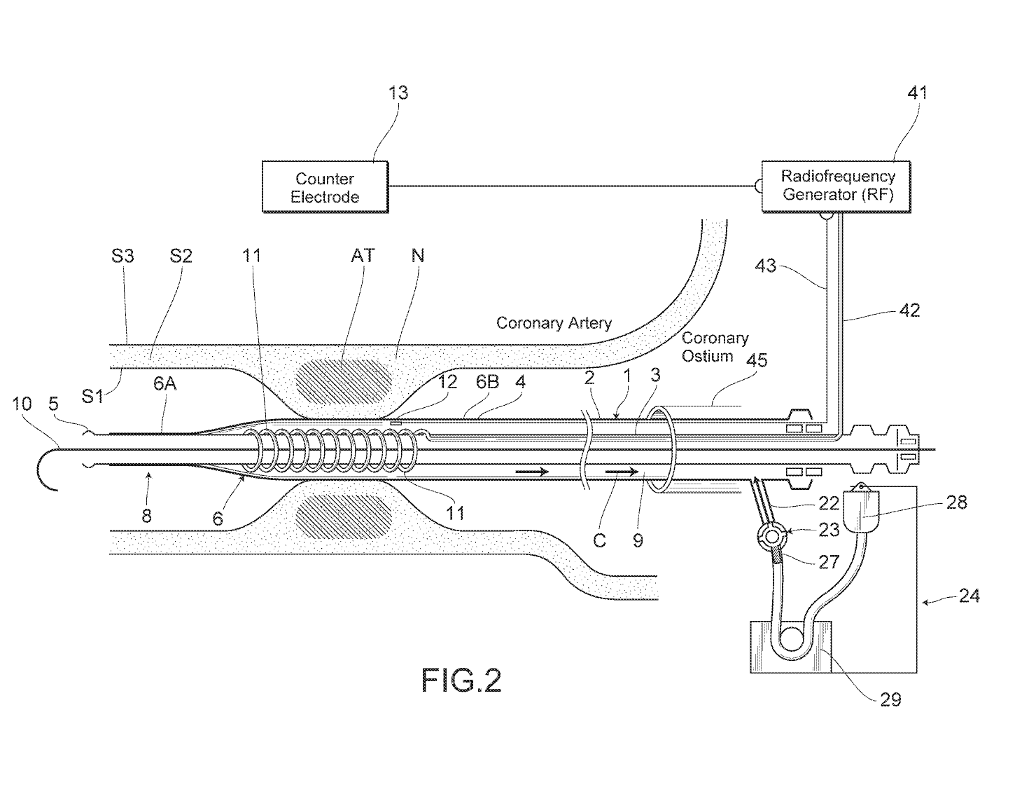

As to a method for implementing the above-discussed configuration, next is a description of the dilation procedures of coronary artery stenosis using the radiofrequency balloon catheter system according to the present embodiment with reference to FIGS. 2 to 4. In each of these figures, symbols S1, S2, and S3 respectively denote the intima, media and adventitia of a coronary artery. Symbol N denotes an artery stenosis site and symbol AT denotes atheroma. Here, FIGS. 1A to 1D should also be referred to because some anatomies are not illustrated.

Into the vicinity of a coronary ostium is intra-arterially inserted a guide sheath 45 through which the balloon catheter, including the catheter shaft 1 and the balloon 6, is further inserted into the coronary artery using the guide wire 10. At the posterior end of the catheter shaft 1, the syringe 25 is connected to the three-way cock 23 connected to the outlet of the solution transport path 9 that is communicated with the inside of the balloon 6 so as to bring the syringe 25 and the solution transport path 9 in communication with each other. Under that condition, if the plunger 31 is pulled back to strongly suction the inside of the balloon 6, the check valve 8 made up of the anterior portion of the neck 6A and the inner tube shaft 3, is closed, thereby turning the inside of the balloon into a negative pressure, thus causing the balloon to be strongly deflated. As a result, the balloon 6 is allowed to be inserted into the artery stenosis site N, as illustrated in FIG. 2.

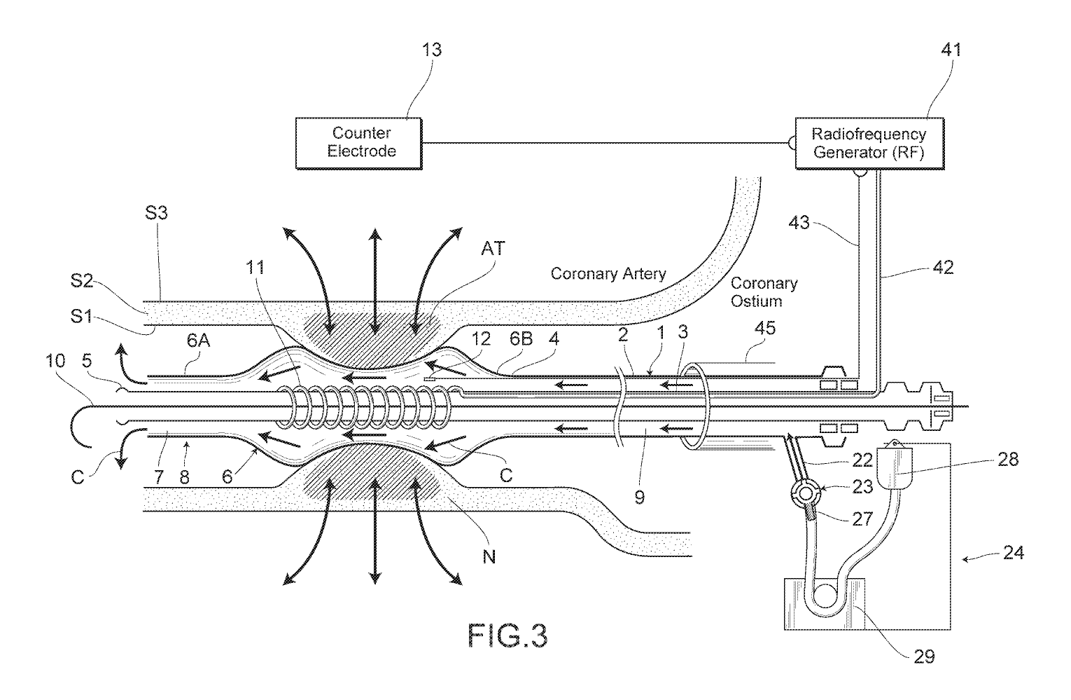

Next, as illustrated in FIG. 3, with the liquid transfusing pump 29 being connected to the communication tube 22 in communication with the solution transport path 9 such that the liquid transfusing pump 29 and the solution transport path 9 is brought into communication with each other through the three-way cock 23, there is initiated a delivery of radiofrequency current between the counter electrode 13, placed on the surface of a body, and the electrode 11 for delivery of radiofrequency current provided within the balloon 6, using the radiofrequency generator 41, while the coolant C is being slowly injected into the balloon. Here, when injection rate of the coolant C is raised, the balloon 6 is inflated so that the check valve 8 is opened, thus allowing the coolant C to be discharged to the outside of the balloon 6. If the artery stenosis site N, being in contact with the outer surface of the balloon 6, is not sufficiently dilated, then, the discharge rate of the coolant C is further increased to thereby elevate the internal pressure of the balloon 6, or otherwise, radiofrequency output of the radiofrequency generator 41 is powered up in order to enhance the intensity of the electric field between the counter electrode 13 and the electrode 11 for delivery of radiofrequency current.

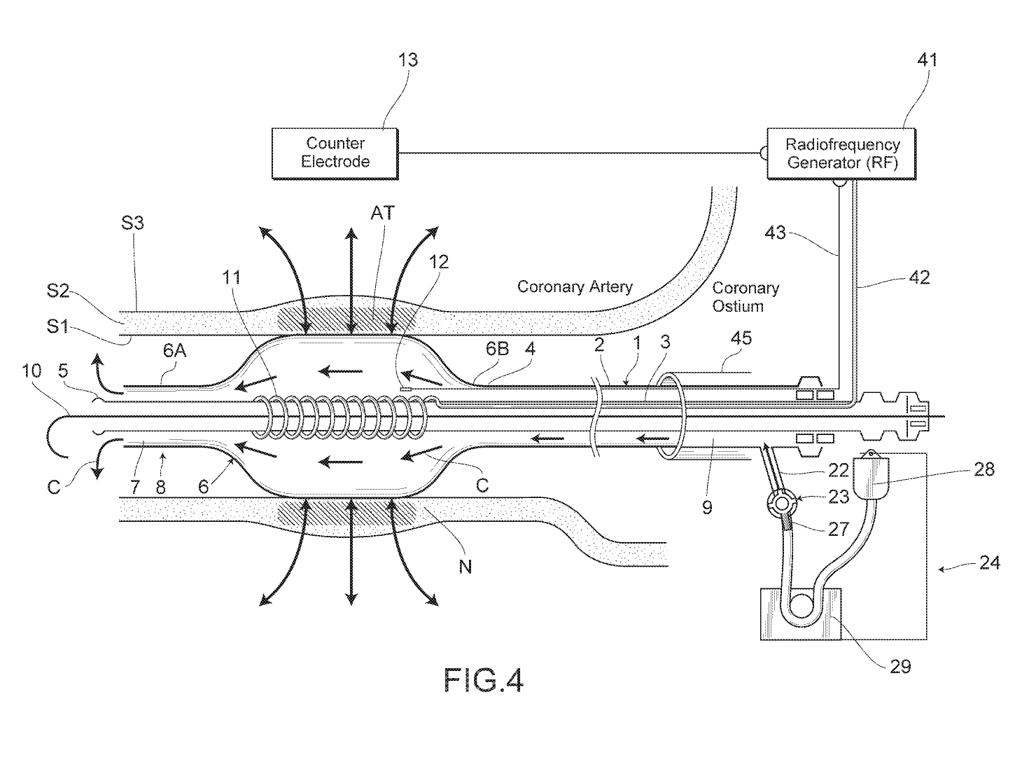

In this way, as illustrated in FIG. 4, if the artery stenosis site N becomes sufficiently dilated, the radiofrequency generator 41 stops delivering the radiofrequency current, and then the coolant C, serving as an in-balloon fluid, is suctioned from the solution transport path 9 using the syringe 25 again to deflate the balloon 6, which is then removed out of the artery stenosis site N. After that, there will be performed a contrast study by way of the tip end of the catheter.

The radiofrequency balloon catheter system according to the present embodiment may be used not only for treatment of artery stenosis as explained above but also for treatments of, e.g., renal-artery stenosis and cerebral artery stenosis, or any other vascular stenoses which may occur all over the body. This system may also be applicable to treatment of stenoses at urethra, ureter, bile passage, or pancreas duct.

In summary, radiofrequency balloon catheters do not cause acute obstruction associated with vascular dissociation or recoil because angioplasty is performed while heating and dilating the stenosis site. Nevertheless, there still has a complication risk of restenosis associated with intimal proliferation. In order to prevent damages to an intima of a blood vessel, there have been proposed various balloon cooling methods in the past, but operability and performance thereof are not necessarily sufficient.

Then, according to the present invention, as described in regard to the embodiment of the present invention, the anterior neck 6A of the balloon 6 constituting the radiofrequency balloon catheter is provided in a manner covering the inner tube shaft 3 so as to let both of them come close to each other to allow a communication path to the outside of the balloon to have a function as a check valve. Accordingly, without the need to change the profile thereof, inflation/deflation of the balloon 6 as well as discharge of the liquid inside the balloon 6 is allowed to be easily performed, thereby achieving enhanced performance and operability. That is, when the inside of the balloon 6 is suctioned by the syringe 25, the check valve 8 gets closed, thus turning the pressure therein to negative. As the result, the balloon 6 gets deflated, enabling the same to easily pass through the stenosis site. When the coolant C is injected, by the liquid transfusing unit 24, into the balloon 6 in order to inflate the same, the check valve 8 is opened to allow the in-balloon solution to be discharged to the outside of the balloon 6, thereby allowing the balloon 6 to be forcibly cooled. When a radiofrequency electric field is radiated from the electrode 11 for delivery of radiofrequency current arranged within the balloon 6, an arteriosclerosis site is heated and melted but the intima thereof remains protected by the cooling of the balloon 6. By enhancing the internal pressure within the balloon 6, stenosis sites get easily dilated without causing any dissection of the vessel.

As is apparent from the above, the radiofrequency balloon catheter system as proposed in the present embodiment has the catheter shaft 1 made up of the inner tube shaft 3, serving as an inner tube, and the outer tube shaft 2, serving as an outer tube, in which both of them are slidable with each other. Between the distal end 5 of the inner tube shaft 3 and the distal end 4 of the outer tube shaft 2 is provided the resilient balloon 6 that is inflatable and deflatable. The anterior neck 6A of the balloon 6 covers the inner tube shaft 3 to thereby define the check valve 8 such that the clearance gap therebetween is open if the balloon 6 is subject to a positive pressure, while it is closed as they are arranged in contact with each other if the balloon 6 is subject to a negative pressure. Also, within the balloon 6 is arranged the electrode 11 for delivery of radiofrequency current, which is connected to the radiofrequency generator 41 via the electric wire 42 in the catheter shaft 1. The solution transport path 9 that is defined by the outer tube shaft 2 and the inner tube shaft 3 and is in constant communication with the inside of the balloon 6, is connected to the liquid transfusing pump 29 serving as a liquid feed pump for feeding the coolant C.

The above-described schematic configurations are illustrated in FIG. 1A. Further, as illustrated in FIG. 1B, when the coolant C inside the balloon 6 is suctioned through the catheter shaft 1, the check valve 8 defined by the anterior neck 6A of the balloon 6 and the inner tube shaft 3 is closed, thus turning the pressure inside the balloon 6 to negative. Also, as illustrated in FIG. 2, when the coolant C inside the balloon 6 is suctioned, the balloon 6 is deflated and thus inserted into the artery stenosis site N.

As illustrated in FIG. 3, when the coolant C is injected into the balloon 6, the balloon 6 becomes inflated to cause the check valve 8 to be opened, letting the coolant C be discharged to the outside of the balloon 6, thereby cooling the balloon 6 as the balloon 6 itself serves as a path for the coolant C. Discharge rate of the coolant C depends on injection rate of the coolant to be injected into the balloon 6, and further on the elasticity and/or shape of the anterior neck 6A of the balloon 6 serving as a valving element. Further, an “overlap” between the anterior neck 6A of the balloon, in communication with the outer tube shaft 2, and the inner tube shaft 3 define a check valve 8 that allows adjustment of the discharge rate of the coolant C from the balloon 6 by sliding the inner tube shaft 3 against the outer tube shaft 2 to change the extent of the “overlap”, as shown in FIGS. 1C and 1D. Concurrently therewith, upon delivery of radiofrequency current, a radio frequency electric field is radiated uniformly from the electrode 11 for delivery of radiofrequency current arranged inside the balloon 6, and the internal pressure of the balloon 6 is elevated, thereby allowing the balloon to dilate the stenosis site N while heating the same. In the meantime, the intima is protected against heating by the cooling of the balloon, as illustrated in FIG. 4. Accordingly, there can be provided an excellent radiofrequency balloon catheter system enabling a balloon catheter thereof to easily pass through a stenosis and dilate the stenosis while heating the same, with an intima S1 being protected against damage.

Next, there will be described other various preferred modifications to the above-described radio frequency balloon catheter system.

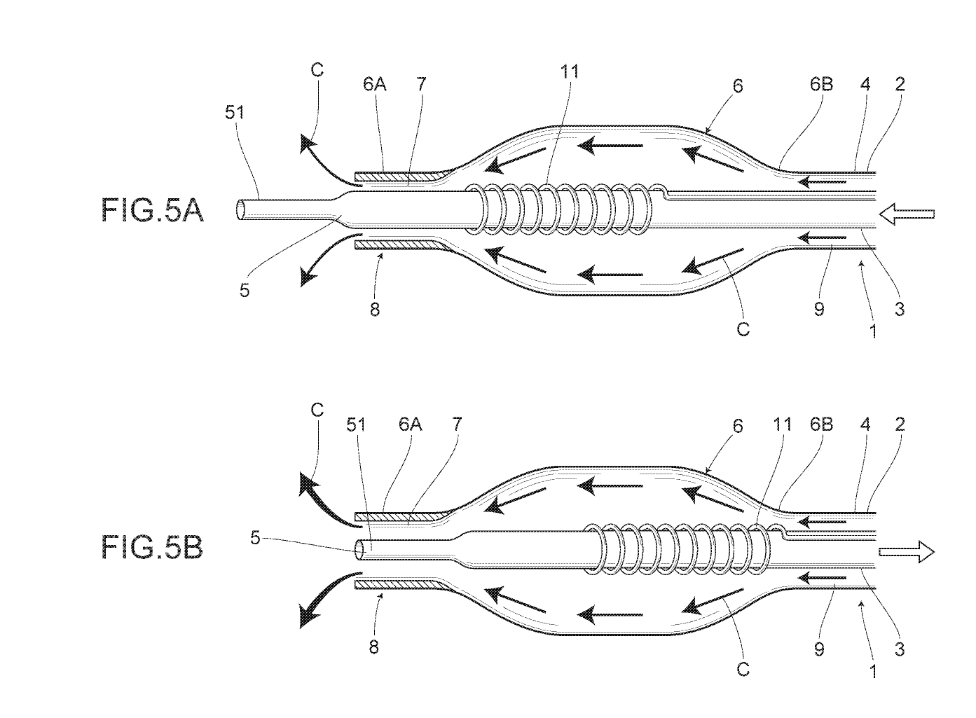

FIGS. 5A and 5B illustrate a modified embodiment in which the outer shape of the inner tube shaft 3 is partially reduced. Particularly, the distal portion 5 of the inner tube shaft 3 has such an outer shape that is not uniformly shaped along the axial direction; i.e., the distal portion 5 thereof is formed with a reduced diameter portion 51 having its distal side narrower in diameter than the proximal side thereof. The other configurations are identical with those in the above embodiment.

According to this modification, the distal portion 5 of the inner tube shaft 3 is reduced in size in a tapered manner to have such a reduced diameter portion 51 that allows the clearance gap 7 between the inner tube shaft 3 and the anterior neck 6A of the balloon 6 to be adjustable in size by sliding the inner tube shaft 3 against the outer tube shaft 2.

Consequently, if the inner tube shaft 3 slides forward against the anterior neck 6A of the balloon 6 to arrange an outer surface of the inner tube shaft 3, positioned more proximal than the reduced diameter portion 51, to be faced against the inner surface of the anterior neck 6A of the balloon 6, then, the clearance gap 7 between the inner tube shaft 3 and the anterior neck 6A of the balloon 6 become narrower, thereby decreasing the discharge rate of the coolant C passing through the check valve 8 from inside of the balloon 6, as illustrated in FIG. 5A. In contrast, as illustrated in FIG. 5B, if the inner tube shaft 3 slides backward against the anterior neck 6A of the balloon 6 to arrange the outer surface of the reduced diameter portion 51 of the inner tube shaft 3 to be faced against inner surface of the anterior neck 6A of the balloon 6, then, the clearance gap 7 between the inner tube shaft 3 and the anterior neck 6A of the balloon 6 become wider, thereby increasing the discharge rate of the coolant C. As illustrated above, discharging rate of the coolant C from the balloon 6 may be easily adjusted by simply sliding the inner tube shaft 3 when the liquid transfusing pump 29 is activated to pump the coolant C into the solution transport path 9.

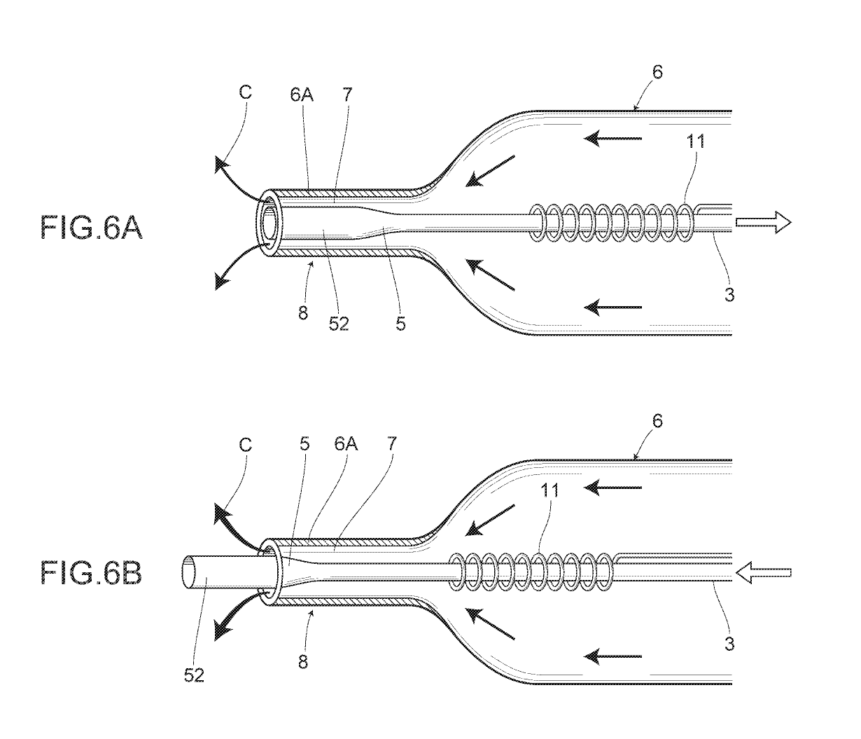

FIGS. 6A and 6B illustrate a modified embodiment in which the outer shape of the inner tube shaft 3 is partially enlarged. Particularly, the distal portion 5 of the inner tube shaft 3 is formed with an enlarged diameter portion 52 having its distal side larger in diameter than the proximal side thereof. The other configurations are identical with those in the above embodiment.

According to this modification, the distal portion 5 of the inner tube shaft 3 is enlarged in size to have such an enlarged diameter portion 52 that allows the clearance gap 7 between the inner tube shaft 3 and the anterior neck 6A of the balloon 6 to be adjustable in size by sliding the inner tube shaft 3 against the outer tube shaft 2.

Consequently, if the inner tube shaft 3 slides backward against the anterior neck 6A of the balloon 6 to arrange the outer surface of the enlarged portion 52 of the inner tube shaft 3 to be faced against the inner surface of the anterior neck 6A of the balloon 6, then, the clearance gap 7 between the inner tube shaft 3 and the anterior neck 6A of the balloon 6 become narrower, thereby decreasing the discharge rate of the coolant C, as illustrated in FIG. 6A. In contrast, as illustrated in FIG. 6B, if the inner tube shaft 3 slides backward against the anterior neck 6A of the balloon 6 to arrange the outer surface of the inner tube shaft 3, positioned more proximal than the enlarged diameter portion 52, to be faced against the inner surface of the anterior neck 6A of the balloon 6, then, the clearance gap 7 between the inner tube shaft 3 and the anterior neck 6A of the balloon 6 become wider, thereby increasing the discharge rate of the coolant C. In this case again, discharging rate of the coolant C from the balloon 6 may be easily adjusted by simply sliding the inner tube shaft 3 when the liquid transfusing pump 29 is activated to pump the coolant C into the solution transport path 9.

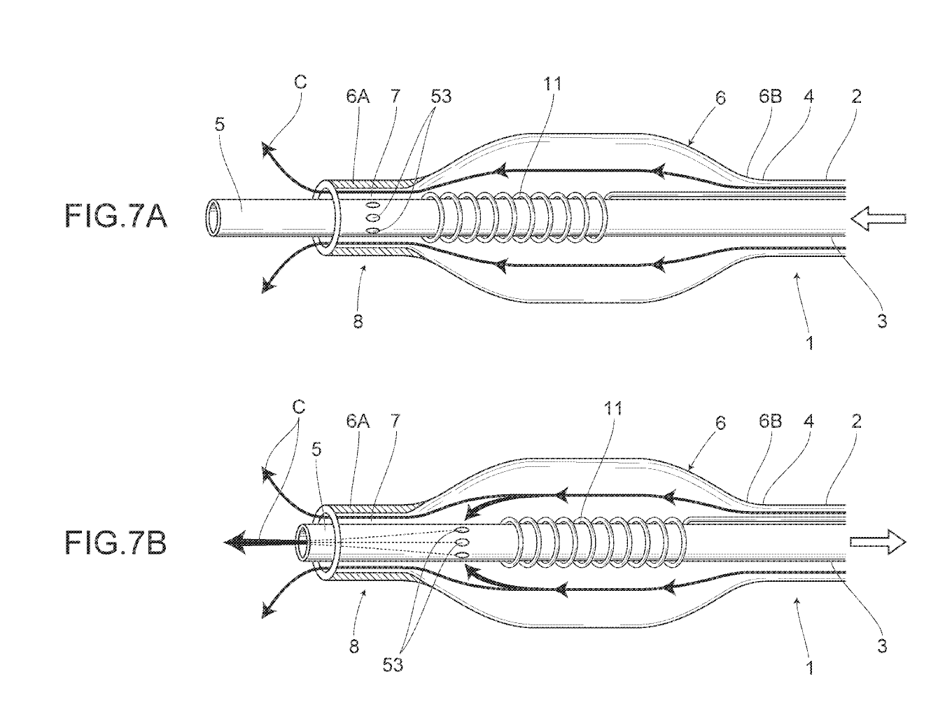

FIGS. 7A and 7B illustrate a modified embodiment in which the distal portion of the tube shaft 3 of hollow shape is perforated with small holes 53. Particularly, the inner tube shaft 3 herein has an outer shape that is uniform along the axial direction, and the distal portion 5, provided as a remote portion thereof, is arranged more distal than the electrode 11 for delivery of radiofrequency current, and is overlapped with the anterior neck 6A of the balloon 6 where the distal portion 5 is perforated with small holes 53 serving as a plurality of holes. The small holes 53 may have various shapes that are not limited to oval or round shapes. The other configurations are identical with those in the above embodiment.

According to this modified embodiment, the distal portion 5 of the inner tube shaft 3 has small holes 53 to adjust the discharge rate of a coolant C from the inside of the balloon 6 by sliding the inner tube shaft 3 against the outer tube shaft 2 to change the extent of the overlap between the small holes 53 of the distal portion 5 of the inner tube shaft 3 and the anterior neck 6A of the balloon 6.

Consequently, if the inner tube shaft 3 slides forward to arrange the small holes 53 of the inner tube shaft 3 to be faced against the inner surface of the anterior neck 6A of the balloon 6, then, the anterior neck 6A of the balloon 6 covers the small holes 53 to thereby pump out the coolant C inside the balloon 6 exclusively through the clearance gap 7 between the inner tube shaft 3 and the anterior neck 6A of the balloon 6, thus decreasing the discharge rate of the coolant C, as illustrated in FIG. 7A. In contrast, as illustrated in FIG. 7B, if the inner tube shaft 3 slides backward to arrange the small holes 53 of the inner tube shaft 3 posterior to the anterior neck 6A of the balloon 6, then, the small holes 53 become cleared of the anterior neck 6A of the balloon 6 to let the holes 53 in direct communication with the inside of the balloon 6 to thereby allow the coolant C inside the balloon 6 to be send out not only through the clearance gap 7, between the inner tube shaft 3 and the anterior neck 6A of the balloon 6, but also through each of the holes 53 into the hollow portion of the inner tube 3 and further to the outside of the balloon 6, thereby increasing the discharge rate of the coolant C. Again, discharging rate of the coolant C from the balloon 6 may be easily adjusted by simply sliding the inner tube shaft 3 when the liquid transfusing pump 29 is activated to pump the coolant C into the solution transport path 9.

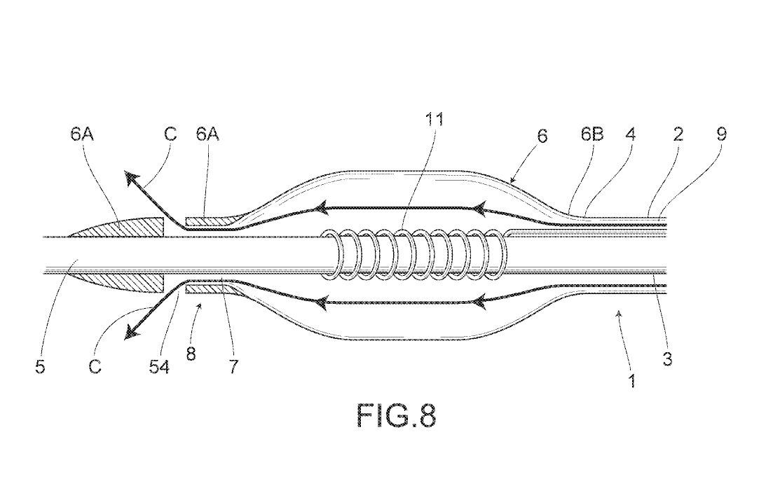

FIG. 8 illustrates a modified embodiment in which the anterior neck 6A of the balloon 6, having a hollow shape, is formed with slits 54. The number of slits is not limited to one, but may be plural. Further small holes may be provided in place of such slits 54. The other configurations are identical with those in the above embodiment.

According to this modified embodiment, the anterior neck 6A of the balloon 6 is designed to have slits 54 or otherwise perforated with small holes to easily open the clearance gap 7 between the anterior neck 6A and the inner tube shaft 3 to communicate with the outside of the balloon 6 when the balloon 6 is subject to a positive internal pressure.

In this case, by virtue of the provision of the slits 54 at the anterior neck 6A of the balloon 6, the coolant C may be easily pumped out through the clearance gap 7 between the anterior neck 6A and the inner tube shaft 3 when the balloon 6 is subject to a positive internal pressure to thereby enhance the cooling capacity thereof.

FIG. 9 illustrates another modified embodiment where electrodes 15a and 15b and a pressure sensor 16 are incorporated into the system in addition to the temperature sensor 12. As shown in this figure, both the temperature sensor 12 and the pressure sensor 16 are provided at a distal portion of the inner tube shaft 3 within the balloon 6 so as to enable temperature measurement of the solution within the balloon 6, and internal pressure measurement inside of the balloon 6, Furthermore, outside the balloon 6, there are provided the electrodes 15a and 15b that are respectively arranged on the distal portion 5 of the inner tube shaft 3 and in the vicinity of the distal end portion 4 of the outer tube shaft 2.

Outside the balloon shaft 1 are arranged an electric impedance measuring potential amplifier 61, a radiofrequency filter 62 and a pressure gauge 63. The electric impedance measuring potential amplifier 61 is connected to the electrodes 15a and 15b, arranged at the front and rear of the balloon 6, respectively through the electric wires 65 and 66, allowing a weak current to flow between the electrodes 15a and 15b, thereby measuring an electric impedance obtained from the voltage value at that time as an electric impedance thereof surrounding the balloon 6, thereby providing the same with a function serving as an electric impedance measuring equipment. Further, the electric impedance measuring potential amplifier 61 has a function to serve as an amplifier for amplifying a far-field potential obtained from the electrodes 15a and 15b and recording that potential, thereby tracking the abrasion progress of the target tissue through monitoring the changes in the electric impedance and potential waveform. Also, the radiofrequency filter 62 is incorporated into the electric circuit for measurement that is composed of the electrodes 15a and 15b, the electric impedance measuring potential amplifier 61 and the electric wires 65, 66 in order to eliminate the influence of the radiofrequency noise generated from the radiofrequency generator 41. In the same way as the foregoing electric wires 42, 43, the electric wires 65, 66 are fixed along the inner tube shaft 3 over the entire axial length of the inner tube shaft 3.

Further, inside the balloon 6 is provided a pressure sensor 16 that outputs detection signals in response to the pressure received on its input surface, and is electrically connected to a pressure gauge 63 through an electric wire 68 provided within the catheter shaft 1. The electric wire 68 is fixed along the inner tube shaft 3 over the whole length of the inner tube shaft 3 extending in an axial direction thereof. As illustrated in FIG. 9, the electric wire 68 is provided outside the electrode 11 for delivery of radiofrequency current. Alternatively, the electric wire 68 may be interposed in the electrode 11 for delivery of radiofrequency current that is provided in a coiled fashion.

The pressure gauge 63 is configured to measure, through detection signals sent out from the pressure sensor 16 via the electric wire 68, a pressure applied from the balloon 6 to a target site, that is, a pressing force, as a degree of pressure applied from the balloon 6 against the target tissue, and then to display the measured pressure. The pressure gauge 63 is arranged outside the balloon catheter 21 along with the radiofrequency generator 41. Preferably, the electric impedance measuring potential amplifier 61 and the radiofrequency generator 41 may be electrically connected with each other so as to allow the measurement outcomes of electric impedance or potential waveform, measured by the electric impedance measuring potential amplifier 61, to be taken into the radiofrequency generator 41. Moreover, the pressure gauge 63 and the radiofrequency generator 41 may be configured to be electrically connected with each other so as to allow the measurement outcomes of pressure, measured by the pressure gauge 63, to be taken into the radiofrequency generator 41. In that case, the radiofrequency generator 41 enables a centralized administrative monitoring of not only a temperature of the balloon 6 and a period of an energization to the electrode 11 for delivery of radiofrequency current, as a monitoring devise, but also an electric impedance around the balloon 6, waveforms of the electric potentials, and a pressing force from the balloon 6 against the tissue. The present embodiment shares common features with the foregoing embodiments except the features described above.

Then, when the balloon 6 is in a state of being inflated, the surrounding space of the pressure sensor 16 is filled with the coolant C while the stream of the coolant C is constantly flowing through the clearance gap toward the outside of the balloon 6. Nevertheless, the directional pressure sensor 16 is hardly affected by the pressure associated with such stream of the coolant C. The pressing forces developed when pressing the balloon 6 against the target site, e.g., vascular stenosis site N, are to be transmitted from the front surface of the membrane of the balloon 6 to the input surface of the pressure sensor 16 via the coolant C provided there inside. For this reason, the pressure sensor 16 becomes highly directive, thereby allowing one to accurately monitor the pressing force from the balloon 6 against the tissue without being influenced by the stream of coolant C inside the balloon 6.

Further, detection signals from the temperature sensor 12 are sent through the electric wire 43 to the radiofrequency generator 41 provided with a thermometer or temperature meter. In response to this, the radiofrequency generator 41 allows monitoring of the internal temperature inside the balloon 6 along with the monitoring results of the pressure sensor 16, thereby enabling one to make sure the effectiveness of the ablation against the target tissue.

Further, the electric impedance measuring potential amplifier 61 allows a weak electric current to flow across the electrodes 15a and 15b via the electric wires 65 and 66 to thereby monitor the electric impedance and far-field potential around the balloon 6, thereby enabling tracking of the ablation progress against the target tissue.

That is, according to this modification, there may be arranged the temperature sensor 12 and the pressure sensor 16 within the balloon 6, such that the temperature sensor 12 may be connected via the electric wire 43 to the radiofrequency generator 41 including the temperature measurement device, while the pressure sensor 16 may be connected to the pressure gauge 63, serving as a pressure measurement device, through another electric wire 68. Hence, there can be monitored a temperature within the balloon 6 and a pressing force of the balloon 6 against tissues, thus enabling one to make sure the effectiveness of ablation against the target tissue.

Further, according to this modified embodiment, there are provided the electrodes 15a and 15b on the anterior and posterior portions of the balloon 6 on the catheter shaft 1, in which the electrodes 15a and 15b are connected via the electric wires 65, 66 to the electric impedance measuring potential amplifier 61 serving as an impedance measurement device. Owing to these electrodes 15a and 15b being arranged in the anterior and posterior portions of the balloon 6, there can be monitored an impedance around the balloon 6, thereby enabling tracking of the ablation progress against the target tissue.

The present invention shall not be limited to the embodiments described above, and various modified embodiments are possible within the scope of the present invention. The radiofrequency balloon catheter system of the present invention can be used not only for dilation of stenosis sites in blood vessel and bile passage, but also in other hollow organs such as urethra, ureter, pancreas duct, trachea, esophagus. Further, the catheter shaft 1 and the balloon 6 may have other various shapes conforming to the sites to be treated, and shall not be limited to those described in the foregoing embodiments.

### DESCRIPTION OF THE REFERENCE NUMERAL

1 catheter shaft
2 outer tube shaft (outer tube)
3 inner tube shaft (inner tube)
6 balloon
6A anterior neck
8 check valve
9 solution transport path
11 electrode for delivery of radiofrequency current
12 temperature sensor
15a,15b electrodes
16 pressure sensor
29 liquid transfusing pump (liquid feed pump)
41 radiofrequency generator (temperature meter)
42 electric wire
43 electric wire
51 reduced diameter portion
52 enlarged diameter portion
53 small holes
54 slits
63 pressure gauge (pressure measurement device)
65 electric wire
66 electric wire
68 electric wire

1. A radiofrequency balloon catheter system comprising:
a catheter shaft comprising an inner tube and an outer tube that are slidable with each other;
a resilient balloon that is inflatable and deflatable and provided between distal ends of the inner tube and the outer tube, said balloon including an anterior neck covering the inner tube;
a check valve defined by the inner tube and the anterior neck covering the inner tube, said anterior neck and said inner tube defining a clearance gap that opens when the balloon is subject to a positive internal pressure, while the anterior neck and the inner tube are contacted with each other to thereby close the clearance gap when the balloon is subject to a negative internal pressure;
an electrode for delivery of radiofrequency current provided within the balloon;
a radiofrequency generator connected to the electrode for delivery of radiofrequency current via a connecting wire within said catheter shaft; and
a solution transport path defined by the outer tube and the inner tube, said solution transport path being in communication with an inside of the balloon, and connected to a liquid feed pump for feeding a coolant,
wherein a distal portion of the inner tube is perforated with small holes to adjust the discharge rate of a coolant from the inside of the balloon by sliding the inner tube against the outer tube to change the extent of an overlap between the anterior neck and the small holes of the distal portion of the inner tube.

2-5. (canceled)

6. The radiofrequency balloon catheter system according to claim 1, wherein a temperature sensor and a pressure sensor are installed within said balloon and are respectively connected to a temperature measurement device and a pressure measurement device via a connecting wire.

7. The radiofrequency balloon catheter system according to claim 1, wherein electrodes are respectively installed in front and back of said balloon on said catheter shaft, and the electrodes are connected to an impedance measurement device via connecting wires.

8. The radiofrequency balloon catheter system according to claim 1, wherein the small holes perforated in the inner tube are positioned more distal to the electrode for delivery of radiofrequency current.

9. The radiofrequency balloon catheter system according to claim 8, wherein the small holes perforated in the inner tube are positioned at a proximal side of said distal portion of the inner tube.

10. The radiofrequency balloon catheter system according to claim 1, wherein the coolant inside the balloon is send out through the clearance gap to the outside of the balloon if the small holes are faced against the anterior neck, while the coolant inside the balloon is send out not only through the clearance gap but also through the small holes into a hollow portion of the inner tube, and further to the outside of the balloon if the small holes are cleared of the anterior neck.

11. The radiofrequency balloon catheter system according to claim 10, wherein said small holes faces against the anterior neck if the inner tube sides forward against the outer tube, while the small holes are cleared of the anterior neck if the inner tube slides backward against the outer tube.

12. The radiofrequency balloon catheter system according to claim 8, wherein the coolant inside the balloon is send out through the clearance gap to the outside of the balloon if the small holes are faced against the anterior neck, while the coolant inside the balloon is send out not only through the clearance gap but also through the small holes into a hollow portion of the inner tube, and further to the outside of the balloon if the small holes are cleared of the anterior neck.

13. The radiofrequency balloon catheter system according to claim 9, wherein the coolant inside the balloon is send out through the clearance gap to the outside of the balloon if the small holes are faced against the anterior neck, while the coolant inside the balloon is send out not only through the clearance gap but also through the small holes into a hollow portion of the inner tube, and further to the outside of the balloon if the small holes are cleared of the anterior neck.
